(19) United States
(12) Patent Application Publication
(10) Pub. No.: US 2022/0040194 A1
(43) Pub. Date: Feb. 10, 2022

(54) USE OF OLANZAPINE FOR TREATMENT OF PARP-INHIBITOR-INDUCED NAUSEA

(71) Applicant: Starton Therapeutics, Inc., Paramus, NJ (US)

(72) Inventors: Pedro Lichtinger Waisman, New York, NY (US); Cidnee Vaykovich, Brooklyn, NY (US); Fotios Plakogiannis, Whitestone, NY (US)

(21) Appl. No.: 17/415,279

(22) PCT Filed: Dec. 17, 2019
(86) PCT No.: PCT/US2019/066916

§ 371 Date: Jun. 17, 2021

Related U.S. Application Data
(60) Provisional application No. 62/780,659, filed on Dec. 17, 2018.

(51) Int. Cl. A61K 31/551; A61K 45/06

(52) U.S. Cl. CPC A61K 31/551; A61K 9/0053; A61K 45/06

(57) ABSTRACT

The present invention generally relates to a combination therapy of a PARP-inhibitor and olanzapine. More particularly, embodiments relate to a method of administering to a patient one or more of a PARP-inhibitor in a therapeutic amount to a patient in need thereof, and olanzapine, in a sufficient amount to treat or alleviate PARP-inhibitor induced nausea or vomiting. In embodiments, the PARP-inhibitor and olanzapine are administered in a common administration schedule. In some embodiments, both actives are orally administered. In other embodiments, olanzapine is transdermally administered. In certain embodiments, the PARP-inhibitor is one or more of olaparib, rucaparib, and niraparib.

### FIELD OF THE INVENTION

The present invention generally relates to a combination therapy of a PARP-inhibitor and olanzapine. More particularly, embodiments relate to a method of administering to a patient one or more of a PARP-inhibitor in a therapeutic amount to a patient in need thereof, and olanzapine, in a sufficient amount to treat or alleviate PARP-inhibitor induced nausea or vomiting. In embodiments, the PARP-inhibitor and olanzapine are administered in a common administration schedule. In some embodiments, both actives are orally administered. In other embodiments, olanzapine is transdermally administered. In certain embodiments, the PARP-inhibitor is one or more of olaparib, rucaparib, and niraparib.

### BACKGROUND

PARP inhibitors are a group of pharmacological inhibitors of the enzyme poly ADP ribose polymerase (PARP). PARP-inhibitors are used for multiple indications, most notably including the treatment of cancer; for example, olaparib is a PARP inhibitor used for ovarian cancer. Besides use in the therapy of various types of cancers, PARP-inhibitors may be beneficial in other various other diseases, e.g. acute life-threatening diseases such as stroke and myocardial infarction, and long-term neurodegenerative diseases. Examples of PARP-inhibitors include olaparib, rucaparib, niraparib, and various other small organic compounds that generally share a benzamide moiety.

Patients treated with PARP-inhibitors frequently suffer from various adverse effects, including in particular PARP-inhibitor induced nausea, for which there is currently no effective treatment.

Olanzapine is an antipsychotic medication used to treat schizophrenia and bipolar disorder. It is usually classed with the atypical antipsychotics, a newer generation of antipsychotics. Olanzapine has also been investigated for use as an antiemetic, generally in combination with one or more further agents, e.g. to treat nausea and vomiting after administration of the chemotherapeutic cisplatin.

Effectiveness against nausea and vomiting may depend at least in part on the type and cause of nausea, which appear to differ based on the causative or contributing circumstances and/or agents. There remains a need in the art for an effective method to treat PARP-inhibitor induced nausea.

Surprisingly, it has now been found that when a PARP-inhibitor is administered in a common administration scheme together with olanzapine, the adverse effect of nausea and vomiting can be alleviated or avoided. This is especially the case when olanzapine is administered in a common administration scheme as described herein, and particularly if the form of administration is via a transdermal delivery device.

These and other features and advantages of the present invention will be explained and will become apparent to one skilled in the art through the summary of the invention that follows.

### SUMMARY OF THE INVENTION

According to embodiments provided herein is a method of treating nausea or vomiting in a human subject. The method includes administering a therapeutic amount of a PARP-inhibitor to the human subject in need thereof; and administering olanzapine to the human subject in an amount sufficient to treat one or more of nausea and vomiting; wherein administering the PARP-inhibitor and olanzapine are performed as part of a common administration scheme. In further embodiments, the common administration scheme is characterized by administering olanzapine about 1 to about 24 hours before administration of the PARP-inhibitor. In other embodiments, the common administration scheme is characterized by co-administering olanzapine and the PARP-inhibitor within a window of time of 1 hour or less.

According to further embodiments provided herein, is a method of treating nausea or vomiting in a human subject. The method includes administering a PARP-inhibitor to the human subject; administering olanzapine to the human subject; within a first predetermined amount of time, achieving a minimum level of efficacy for treating nausea or vomiting in the human subject; within a second predetermined amount of time, achieving a preferred level of efficacy for treating nausea or vomiting in the human subject; and maintaining the preferred level of efficacy within a predetermined range for treating nausea or vomiting in the human subject for a third predetermined amount of time. In further embodiments, the first predetermined amount of time is less than about 2 hours, less than about 90 minutes, less than about an hour, less than about 45 minutes, or less than about 30 minutes. In yet further embodiments, the second predetermined amount of time is less than about 5 hours, less than about 4 hours, or less than about 3 hours. In yet further embodiments, the third predetermined amount of time is at least 2 days. In other embodiments, the third predetermined amount of time is at least 3 days, 4 days, 5 days, at least 6 days, at least 7 days, at least 8 days, at least 9 days, at least 10 days, at least 11 days, at least 12 days, at least 13 days, or at least 14 days. In further embodiments, the minimum level of efficacy is achieved when the human subject blood serum level of olanzapine is at least 10 ng/ml. In other embodiments, the preferred level of efficacy is achieved when the human subject blood serum level of olanzapine is at least 20 mg/l. In further embodiments, the predetermined range is a blood serum level of olanzapine that is between about 20 ng/ml and about 40 ng/ml.

The foregoing summary of the present invention with the preferred embodiments should not be construed to limit the scope of the invention. It will become apparent to one of ordinary skill in the art that the embodiments of the invention described herein may be further modified without departing from the spirit and scope of the invention, and various illustrative modifications can be found in the detailed description that follows.

### DETAILED SPECIFICATION

The present invention generally relates to the administration of one or more PARP-inhibitor and of olanzapine, in a common administration scheme to treat nausea that is induced by administration of the PARP-inhibitor.

“PARP” as used herein refers to a group of poly (ADP-ribose) polymerase enzymes (PARP). PARP enzymes are activated by DNA damage, in particular, PARP1 and PARP2 enzymes. These enzymes facilitate DNA repair in pathways involving single-strand breaks (SSBs) and base excision repair (BER). All PARP-inhibitors are generally believed to inhibit both PARP1 and PARP2. The suppression of PARP catalytic activity prevents the formation of poly (ADP-ribose) polymers and blocks the binding of NAD+ at the site of DNA damage, ultimately compromising a cell's ability to overcome DNA-dependent damage.

“PARP-inhibitor” as used herein refers to a chemical compound that blocks an enzyme in cells called poly (ADP-ribose) polymerase (PARP). PARP enzymes help repair DNA upon damage. DNA damage may be caused by various things, including exposure to UV light, radiation, certain anticancer drugs, or other substances in the environment. Many PARP-inhibitors share certain structural commonalities, and typically include a benzamide moiety, or a benzamide-derivative moiety, and find use as chemotherapeutic agents directed at targeting cancers with defective DNA-damage repair. Blocking PARP keeps cancer cells from repairing their damaged DNA, thus causing them to die.

Examples of PARP inhibitors include olaparib (AZD-2281, Lynparza® by Astra Zeneca), e.g. for breast, ovarian, colorectal or prostate cancer, rucaparib (PF-01367338, Rubraca® by Clovis Oncology), e.g. for metastatic breast and ovarian cancer, niraparib (MK-4827, Zejula® by Tesaro), e.g. for epithelial ovarian, fallopian tube, and primary peritoneal cancer, talazoparib (BMN-673, originally developed by BioMarin Pharmaceutical Inc., currently in development by Pfizer), e.g. for advanced hematological malignancies and for advanced or recurrent solid tumors and for metastatic germline BRCA mutated breast cancer, veliparib (ABT-888, developed by AbbVie), e.g. for advanced ovarian cancer, triple-negative breast cancer, non-small cell lung cancer (NSCLC), and metastatic melanoma, CEP 9722 for non-small-cell lung cancer (NSCLC), E7016 (developed by Eisai), e.g. for melanoma, BGB-290, iniparib, 3-aminobenzamide (3-AB, a prototypical PARP inhibitor), PJ-34, Nu1085, INO-1001, CEP-8933/CEP-9722, and nicotinamide.

Olanzapine is chemically known as 2-methyl-4-(4-methylpiperazin-1-yl)-5H-thieno[3,2-c][1,5]benzodiazepine (IUPAC). Olanzapine may be used in form of its base or one of its salts, including, without limitation, the following olanzapine salts: hydrochloride, pamoate, malonate, glycolate, maleate, and benzoate.

The compounds for use in the present invention (i.e. the PARP-inhibitor and olanzapine) are administered in a common administration scheme, i.e. the compounds may be administered serially, or at substantially the same time, or simultaneously. This common administration scheme includes administering the compounds separately but at substantially the same time, or administering them at the same time in one pharmaceutical preparation. Further, the common administration scheme includes serial administration, i.e. administering the compounds of the present invention one after the other. In serial administration, preferably, olanzapine is administered before the PARP-inhibitor is administered, for example, at least about 1-24 hours before, preferably at least 6-12 hours before, for example, at least 1, 2, 3, 4, 5, 6, 7, 8, 9, 10, 11, or 12 hours before. Rapid release dosage forms of olanzapine are preferably administered about 1-3 hours prior to PARP-inhibitor administration, or may be administered simultaneously, or at substantially the same time (e.g. within an hour of administration of the PARP-inhibitor).

Therapeutically effective doses of the compounds for use in the present invention will vary depending on what therapeutic effect is desired and the special needs and idiosyncrasies of the individual patient. Accordingly, a wide range of doses for each compound as well as dose ratios of the two compounds are possible, as will be apparent to the person of ordinary skill. The dose for the PARP-inhibitor will also depend on the particular PARP-inhibitor chosen, as will be apparent to a person of ordinary skill. Generally, a suitable dose of olanzapine to treat PARP-induced nausea may be, without limitation, about 1-20 mg daily. Preferably, less than the standard dose may be used, and may avoid sedation side effects of olanzapine. For example, a dose of about 5 mg olanzapine or lower daily may be effective, in particular if a transdermal dosage form is used. For example, about 1-3 mg olanzapine may be used.

Olanzapine is available in various dosage forms including oral tablets (2.5 mg, 5 mg, 7.5 mg, 10 mg, 15 mg, and 20 mg), as short-acting intramuscular (IM) injection (10 mg), and as extended-release suspension via IM injection (210 mg/vial, 300 mg/vial, 405 mg/vial).

PARP-inhibitors are available in various dosage forms. For example, olaparib is available as oral tablets (100 mg, 150 mg) and capsules (50 mg); rucaparib is as oral tablets (200 mg, 250 mg, 300 mg); and niraparib is available as oral capsules (100 mg).

Olanzapine base, its salts and any pharmaceutically acceptable forms thereof, either alone or in combinations, are collectively referred herein as “olanzapine” or “OLA”, unless otherwise stated or apparent from the context. OLA includes e.g., without limitation, olanzapine isomers, racemic forms, solvates, hydrates, hydrates of a racemate, amorphous forms, crystalline forms, co-crystals, solid solutions, isomers, prodrugs, analogs and derivatives.

Acid addition salts of olanzapine that may be suitable to form part of the formulations described herein include, without limitation, hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

In general, compounds for use in the present invention will be administered as pharmaceutical compositions by any one of the following administration routes: oral, systemic (e.g., transdermal, intranasal or by suppository), or parenteral (e.g., intramuscular, intravenous or subcutaneous) administration; inhalation delivers a therapeutic agent via the respiratory tract. Administration may be adjusted according to the administration route, and the degree of the affliction, as will be apparent to a person of ordinary skill.

Oral compositions can take the form of, e.g., tablets, pills, capsules, semisolids, powders, sustained release formulations, solutions, suspensions, elixirs, aerosols, or any other appropriate composition.

Transdermal delivery systems (TDS) include transdermal formulations which may be in form of a liquid or semi-solid form of a desired degree of viscosity, for example, a solution, suspension, dispersion, emulsion, micro emulsion, nano emulsion, gel, ointment, cream, paste, lotion, mousse, or balm. Alternatively the transdermal formulation may form part of a TDS that comprises the transdermal formulation. Exemplary TDS include, without limitation, topical formulations (e.g. for occlusive or non-occlusive application to the skin or mucous membrane), gels, lotions, sprays, metered dose transdermal sprays, aerosols, magma, transdermal patches, monolithic matrix patches with or without adhesive, drug-in-adhesive patches, matrix reservoir patches (with a separate matrix reservoir optionally surrounded by adhesive), hydrogel matrix patches, microneedle systems, iontophoresis systems, or combinations thereof.

In embodiments, the transdermal formulation may be in form of a liquid or gel and may be incorporated in a transdermal patch. For example, without limitation, the transdermal formulation may include a polymer matrix, which may be adhesive or non-adhesive, e.g., without limitation a polyacrylic adhesive. Matrix patches include those with a single matrix layer, or multiple matrix layers.

For the above uses the required dosage will vary depending on the mode of administration, the particular condition to be treated and the effect desired. In general, satisfactory results are indicated to be obtained systemically at daily dosages in a larger mammal, e.g. humans, in the ranges indicated below for the oral dosage forms, which may be administered in a single daily dose, in particular an extended/retarded-release dose, or in divided doses, e.g. 2, 3, or 4 times a day. For the PARP-inhibitor, a suitable daily dosage may be from about 50 mg to about 750 mg of the active, typically administered e.g. twice daily. For example, for olaparib or rucaparib, a suitable daily dosage may be from about 150 mg to about 750 mg, e.g. about 300 to about 600 mg of the active, e.g. in form of an oral dosage form; and for niraparib, a suitable daily dosage may be from about 50 mg to about 400 mg, e.g. about 100 to about 300 mg of the active, e.g. in form of an oral dosage form. For olanzapine, a suitable daily dosage may be from about 1 to about 20 mg, e.g. about 2.5 to about 10 mg of the active per day, e.g. in form of an oral or transdermal dosage form.

Olanzapine transdermal patches that may be suitable for use in the methods of the invention have been previously described, e.g. in U.S. Publication No. 20070148218, U.S. Pat. No. 5,891,461, and by Sharma & Aggarwal et al. 2010; Der Pharmacia Lettre, 2010, 2(6): 84-98; these are incorporated by reference herein in their entirety.

The amount of OLA active in a transdermal formulation or patch depends on the transdermal vehicle chosen, as will be apparent to a person of ordinary skill. In case of a matrix patch, the matrix may contain the OLA active in an amount of about 0.5% w/w to about 50% w/w, for example about 1% w/w to about 25% w/w, or about 2% w/w to about 15% w/w. In further embodiments, the amount of OLA active is about 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%.

Matrix-forming or gel-forming polymers may be used to form a transdermal gel, reservoir patch or matrix patch, and a large number of such polymers may be employed alone or in combination in amounts depending on the particular delivery vehicle and intended use (e.g. viscosity, duration of application, adherence etc.) as will be apparent to a person of ordinary skill. Exemplary polymers include, without limitation, cellulose and its derivatives (such as but not limited to hydroxy methyl cellulose, Aquasolve™ hypromellose acetate succinate, hydroxypropyl methyl cellulose, hydroxypropyl cellulose, ethyl cellulose, hydroxyethyl cellulose, methyl cellulose, carboxymethyl cellulose, sodium carboxymethyl cellulose, microcrystalline cellulose blends, cellulose acetate phthalate, propylmethylcellulose phthalate, etc.), biodegradable polymers (such as but not limited to gelatin, chitosan, starch, polyacrylic acid, polyvinyl alcohol, etc.), gums (such as but not limited to guar gum, gum copal, gellum gum, xanthan gum, locust bean gum, gum arabic, tragacanth, cassia gum, karaya gum etc.), polysaccharides (such as but not limited to carrageenan, agar, pectin, mannan, alginic acid, dextran, pullulan, etc.), adhesive polymers (such as but not limited to sorbitol, polyvinyl alcohol, vegetable starch, d-sorbitol, gelatin, etc.), polyvinyl alcohol and its derivatives, polyvinyl pyrrolidone and its derivatives, polyvinylpyrrolidone, polyvinyl pyrrolidone homopolymer and polyvinyl pyrrolidone copolymers (PVP, Poloxamer), crospovidone, pressure sensitive adhesives (PSA), solvent-borne PSA, water-borne PSA, hot melt PSA (e.g. BIO-PSA Hot melt 7-4560, hot melt adhesives based on thermoplastic polymers such as ethylene vinyl acetate copolymers, polyesters, paraffin waxes, polyamides, low density polypropylene, polyurethanes, and/or ethylene ethacrylate copolymers), acrylic PSA, silicone PSA, rubber PSA, PSA containing one or more of a hydroxyl functional group and a carboxyl functional group, PSA not containing functional groups, PSA not containing one or more of a hydroxyl functional group and a carboxyl functional group, acrylate copolymers, acrylic adhesive 788, acrylic acid-isooctyl acrylate copolymer, dimethylaminoethyl methacrylate-butyl methacrylate-methyl methacrylate copolymer, isooctyl acrylate, styrene/isoprene/styrene block copolymer, poly(meth) acrylate polymers (such as but not limited to amino alkyl methacrylate copolymers, methacrylic acid copolymers, methacrylic ester copolymers, ammonioalkyl methacrylate copolymers (e.g. Eudragit® L100-55, Eudragit® E PO, Eudragit® RL, Eudragit E® 100, Plastoid® B, dimethylaminoethyl methacrylate-butyl methacrylate-methyl methacrylate copolymer), styrene rubber block copolymers, elastomers, silicone elastomers, polybutadiene, styrene-butadiene copolymers, dimethiconol/trimethylsiloxysilicate crosspolymer, dimethiconecrosspolymer, hydrogenated polybutene, polybutene, ethylene-propylene copolymers, polyoxyethylenepoloxypropylene block copolymers, cross-linked polyethylene glycol, polyvinyl acetate phthalate, sodium polyacrylate, carboxyvinyl copolymers, carbomer copolymer type B, carbomer homopolymer type c, polyacrylamide, maleic anhydride copolymers, butyl ester of methyl vinyl ether/maleic anhydride copolymer (125000 mw), poly(acrylic acid), polyacrylic acid sodium salt, polyethylene and its copolymers, clays such as silicate, polyacrylate copolymers, isobutylene, natural rubbers, synthetic rubbers (e.g. styrene-diene copolymers, isoprene block copolymers, acrylonitrile butadiene rubber, butyl rubber or neoprene rubber), dimethicone, and polyvinyl acetate.

Exemplary acrylic PSA include, without limitation, one or more of: Duro-Tak® 87-2196, Duro-Tak® 387-2051, Duro-Tak® 87-2194, Duro-Tak® 87-235A, Duro-Tak® 387-2054, Duro-Tak® 87-900A, Duro-Tak® 87-9301, Duro-Tak® 387-2516, Duro-Tak® 387-2510, Duro-Tak® 280-2516, Duro-Tak® 87-4098, GELVA GMS® 788, GELVA GMS® 9073, Duro-Tak® 387-2353, Duro-Tak® 87-2074, Duro-Tak® 387-2287, Duro-Tak® 87-2852, Duro-Tak® 87-2054, GELVA® 737, Duro-Tak® 80-1196, Duro-Tak® 87-2070, Duro-Tak® 87-2979, Duro-Tak® 87-2888, and Duro-Tak® 87-2296. Exemplary silicone PSA include, without limitation, one or more of: BIO-PSA® 7-4401, BIO-PSA® 7-4402, BIO-PSA® 7-4501, BIO-PSA® 7-4502, BIO-PSA® 7-4601, BIO-PSA® 7-4602, (Dow Corning®, Dow Chemicals, Midland Mich.), SRS7-4502, SRS7-4501, SRS7-4602, SRS7-4602, amine compatible silicone PSA, a rubber PSA. Exemplary amine compatible silicone PSA include, without limitation, one or more of BIO-PSA® 7-4101, BIO-PSA® 7-4102, BIO-PSA® 7-4201, BIO-PSA® 7-4202, BIO-PSA® 7-4301, BIO-PSA® 7-4302. Exemplary rubber PSA include, without limitation, one or more of: polyisobutylene of low molecular weight, polyisobutylene of medium molecular weight, polyisobutylene of high molecular weight (including, e.g., polyisobutylene 1100000 MW, 35000 MW, 800000 MW, 55000 MW, 2300 MW, or mixtures thereof), Duro-Tak® 87-6908, and polyisobutylene/polybutene adhesive.

Adhesives that may be particularly suitable for the drug-in-adhesive patches and formulations therefore described herein include, without limitation, high molecular weight or highly crosslinked adhesives, typically available as self crosslinkable acrylic adhesives. Examples of such adhesives include, without limitation, Duro-Tak® 387-2516, Duro-Tak® 387-2051, Duro-Tak® 87-2852, Duro-Tak® 87-2194 and Duro-Tak® 87-2852 self crosslinkable acrylic adhesives (available from National Starch and Chemical Company, 10 Finderne Ave., P.O. Box 6500, Bridgewater, N.J. 08807-0500), and GELVA® 737, GELVA® 2655, and GELVA® 1753 self crosslinkable acrylic adhesives (Monsanto's Chemical Group, 730 Worcester Street, Springfield, Mass. 01151).

Duro-Tak® 387-2516 is an acrylic copolymer adhesive containing EHA, vinyl acetate and hydroxyethyl acrylate and is commercially available from National Starch and Chemical Co, Bridgewater, N.J.). Alternatively, the adhesive may be an acrylic adhesive having one or more of hydroxyl functional groups and carboxyl functional groups. Still alternatively, the acrylic adhesive may be a “nonfunctional” adhesive which does not contain function groups (e.g. lacks —OH groups, —COOH groups, or both). Preferably the acrylic adhesive may be a pressure sensitive adhesive (PSA).

In embodiments, the TDS may comprise one or more optional carriers and excipients, some of which may have dual or multiple functionality, e.g. a particular excipient may function as e.g. a penetration enhancer or as e.g. a plasticizer, or both, depending on concentration, type of transdermal system, and its components. Optional carriers or excipients include, without limitation, solvents, solubilizers, diluents, suspending agents, dispersing agents, gelling agents, polymers, penetration enhancers, plasticizers, pH adjusting agents, buffering agents, pH stabilizers, emulsifying agents, auxiliary emulsifying agents, surfactants, suspending agents, stabilizers, preservatives, antioxidants, chelating agents, complexing agents, emollients, humectants, demulcents, skin irritation reducing agents, antioxidants, oxidants, tackifiers, fillers.

In embodiments, the TDS may comprise a solvent, e.g. one or more of a C1-C20 alcohol (e.g., without limitation, one or more of: methanol, ethanol, isopropyl alcohol, butanol, propanol, 2-methyl-2-propanol, aka t-butyl alcohol, pentanol, 2,4-dimethyl-2-pentanol, 3,5-dimethyl-3-hexanol, and alcohols having C7, C8, C9, C10, C11, C12, C13, C14, C15, C16, C17, C18, C19 or C20 carbon atoms), polyhydric alcohols, glycols (e.g., without limitation: propylene glycol, polyethylene glycol, dipropylene glycol, hexylene glycol, butyene glycol, glycerine), derivatives of glycols, pyrrolidone (e.g., without limitation: N methyl 2-pyrrolidone, 2-pyrrolidone), sulfoxides (e.g., without limitation, dimethyl sulfoxide aka DMSO and decymethylsulfoxide), dimethylisosorbide, mineral oils, vegetable oils, water, polar solvents, semi polar solvents, and non polar solvents.

In embodiments, the TDS may comprise a surfactant, solubilizer, emulsifying agent, or dispersing agent, including anionic, cationic, nonionic and amphoteric surfactants, e.g. one or more of a propylene glycol, monocaprylate type I, propylene glycol monocaprylate type II, propylene glycol dicaprylate, medium chain triglycerides, propylene glycol monolaurate type II, linoleoyl polyoxyl-6 glycerides, oleoyl-polyoxyl-6-glycerides, lauroyl polyoxyl-6-gylcerides, polyglyceryl-3-dioleate, diethylene glycol monoethyl ether, propylene glycol monolaurate type I, polyglyceryl-3-dioleate, caprylocaproyl polyoxyl-8 glycerides, cyclodextrins, Diethylene glycol monoethyl ether (DEGEE), a polysorbate/polyethoxylated sorbitan ester or Tween®-type surfactant, a sorbitan ester or Span®-type solvent surfactant, a glycol, hexylengycol, a Brij® type surfactant, and sodium lauryl sulfate. DEGEE (also known as Di(ethylene glycol) ethyl ether or 2-(2-Ethoxyethoxy)ethanol)) is commercially available e.g. under the various trade names including Transcutol® (TC), Transcutol® P, Transcutol® CG, Transcutol® HP (Gattefosse, Lyon, France), and Carbitol™ (Dow Chemicals, Midland Mich.). The Span® or Tween® surfactant may, without limitation, be selected from one or more of: Span 20®, Span®40, Span® 60, Span®80, Span®83, Span®85, Span®120, Tween 20®, Tween 21®, Tween 40®, Tween 60®, Tween 61®, Tween 65®, and Tween 80®. Brij® is a group of nonionic surfactants commercially available from various sources (e.g. Sigma-Aldrich), and may be selected from one or more of Brij® 93 (average Mn ˜357), Brij® S 100 (average Mn ˜4,670), Brij® 58 (average Mn ˜1124), Brij® 010 (average Mn ˜709, also known as Brij 97, C18-1E10, Polyoxyethylene (10) oleyl ether, C18H35(OCH2CH2)nOH, n˜10), Brij® C10 (average Mn ˜683), Brij® L4 (average Mn ˜362, also known as polyethylene glycol dodecyl ether, polyoxyethylene (4) lauryl ether, (C20H42O5)n), BRIJ® 020 (average Mn ˜1,150, Polyoxyethylene (20) oleyl ether, C18H35(OCH2CH2)nOH, n˜20), Brij® S2 MBAL (also known as Brij® S2, polyethylene glycol octadecyl ether, polyoxyethylene (2) stearyl ether, main component: diethylene glycol octadecyl ether, C18H37(OCH2CH2)2OH), Brij® S10 (average Mn ˜711), Brij® S20, and Brij® 35 (also known as Brij® L23, C12E23, polyoxyethylene lauryl ether, (C2H40)nC₁₂H₂₆O). Suitable amounts of a surfactant to include into transdermal formulations to perform a surfactant function may be less than 5% (wt/wt), typically e.g. less than 4, 3, 2, 1, or 0.5%. Suitable amounts for solvent/solubilizing functions may be from 5% to about 50%. Amounts may be increased or decreased to achieve a suitable and sufficient amount, as will be apparent to a person of ordinary skill in the art.

A glycol is class of small organic compounds (e.g. MW typically below 150), or a polymer thereof, that belongs to the alcohol family, and wherein two hydroxyl (—OH) groups are attached to different carbon atoms. The simplest member of the glycol class is ethylene glycol (also known as 1,2-ethanediol), other members include, without limitation, propylene glycol (also called 1,2-propanediol), butylene glycol (1,3-butanediol), 1,4-butanediol, pentylene glycol, (1,2-Pentanediol), hexylene glycol (2,4-pentanediol), 2-ethyl-1,3-hexanediol, and 2-methyl-2-propyl-1,3-propanediol. Similarly, higher molecular weight polymers of the above glycol diols, in particular of ethylene glycol, may be used; these include, without limitation, polyethyleneglycol (PEG). PEGs are available in different molecular weights, typically from about 300 g/mol to about 10,000,000 g/mol, e.g. PEG 300, 400, 600, 800, 1000, 1500, 6000, 8000, 10,000, 20,000, 35,000, etc. PEGs of different molecular weight have identical or similar surfactant properties but the higher molecular weight polymers may be preferred for their additional thickening function which may be desired in some patch formulations.

In embodiments, the TDS may comprise one or more gelling agents, one or more thickening agents, and one or more suspending agents. Gelling agents, thickening agents and suspending agents may be one or more agent selected from the group of, e.g., without limitation, polymers, natural polymers, polysaccharides and its derivatives (e.g., without limitation, agar, alginic acid and their derivatives, cassia tora, collagen, gelatin, gellum gum, guar gum, pectin, carrageenan, potassium carageenan, sodium carageenan, tragacanth, xanthan gum, gum copal, chitosan, resin), semisynthetic polymers and their derivatives (e.g., without limitation, cellulose and its derivatives; e.g., without limitation, methylcellulose, ethyl cellulose, carboxymethyl cellulose, hydroxylpropyl cellulose, hydroxylpropylmethyl cellulose), synthetic polymers and their derivatives (e.g., without limitation, carboxyvinyl polymers and carbomers; e.g., without limitation, carbopol 940, carbopol 934, carbopol 971p NF), polyethylene and its copolymers, clays (e.g., without limitation, silicates, bentonite, and bentonites), silicon dioxide, polyvinyl alcohol, acrylic polymers (e.g., without limitation, eudragit), acrylic acid esters, polyacrylate copolymers, polyacrylamide, polyvinyl pyrrolidone homopolymer and polyvinyl pyrrolidone copolymers such as but not limited to (PVP, Kollidon 30, poloxamer), isobutylene, ethyl vinyl acetate copolymers, natural rubber, synthetic rubber, pressure sensitive adhesives such as silicone polymers (e.g., without limitation, bio psa 4302, bio-psa 4202), acrylic pressure sensitive adhesives (e.g., without limitation, Duro-tak 87-2156, duro-tak 387-2287), polyisobutylene (e.g., without limitation, polyisobutylene of low molecular weight, polyisobutylene of medium molecular weight, polyisobutylene 35000 mw), acrylic copolymers, rubber based adhesives, hot melt adhesives, styrene-butadiene copolymers, bentonite, water swellable polymers, organic solvent swellable polymers, swellable polymers comprising both water and organic solvent.

In embodiments, the TDS, and in particular a coating formulation for a patch, may comprise volatile solvents which are removed from the patch matrix upon its drying; such volatile solvents include: methanol, ethanol, propanol, 1-propanol, 2-propanol, ethyl acetate, acetone, dichloromethane, chloroform, toluene, and IPA).

Alternatively or additionally, other additional excipients may be added, e.g., without limitation, as detailed below. Typically all optional and additional excipients are added in a smaller amount, e.g., without limitation, less than about 5, 4, 3, 2, 1, 0.5 or 0.1% (% wt after drying). The total of all excipients preferably should be below about 15, 10, or 5% (% wt after drying).

To increase skin permeation and/or penetration of OLA, a permeation enhancer may be included into a formulation for use in methods of the invention. Numerous penetration enhancers that include structurally diverse compounds are known and may be used alone or in combination, as will be apparent to a person of ordinary skill. For example, penetration enhancers may include one or more of alcohols (e.g. ethanol, propanol, and isopropanol), fatty acids, fatty alcohols, fatty acid derivatives, fatty alcohol derivatives, sulfoxides (e.g. decylmethyl sulfoxide), amides (e.g. dimethylformamide, azone, urea, dimethylacetamide), pyrrolidone derivatives (e.g. 1-methyl-4-carboxy-2-pyrrolidone, 1-methyl-2-pyrrolidone, 1-lauryl-4-methoxycarbonyl-2-pyrrolidone), terpenes (e.g. menthol, limonene, terpineol, pinene, carvol), ethyl acetate, methyl acetate, octisalate, pentadecalactone, and acrylamide.

Fatty acid or alcohol permeation enhancers include those wherein the fatty acid, fatty acid derivative, fatty alcohol, or fatty alcohol derivative consists of a substituted fatty acid moiety, or a substituted fatty alcohol moiety, e.g., wherein the fatty acid moiety or the fatty alcohol moiety of the FD enhancer has a carbon chain length from C4 to C26. This may include, for example, without limitation, fatty acid esters, in particular those wherein the fatty acid or fatty acid part moiety has a carbon chain length of C4 to C26, or longer, including in particular C4, C5, C6, C7, C8, C10, C11, C12, C14, C16, C18, C20, C22, C24 and C26, or combinations thereof. For example, one or more of C12-26 fatty acids, alcohols and their derivatives, e.g. C18, may be combined with a shorter chain fatty acid from C4 to C10 (e.g., without limitation, C4, C5, C6, C7, C8, C10). Example derivatives include substituted fatty acids or fatty alcohols, for example as described herein, comprising one or more additional group selected from, without limitation, hydroxyl, ethyl, methyl, propyl, butyl, and glyceryl.

Exemplary particular fatty acid or alcohol permeation enhancers include, without limitation, saturated, unsaturated, monounsaturated and polyunsaturated fatty acids, e.g., without limitation, omega-3, omega-6, omega-7 and omega-9 fatty acids. The saturated, unsaturated, monounsaturated and polyunsaturated fatty acids may include, e.g., without limitation, fatty acids with a carbon chain of C12, C14, C16, C18, C20, C22, C24 and C26, in particular, without limitation, e.g. C14, C16, C18, and C20. Fatty acid or alcohol permeation enhancers further include, e.g., branched-chain saturated fatty acids, including, without limitation, methyl-branched fatty acids, e.g. isostearic acid, and ethyl-branched fatty acids. Fatty acid or alcohol permeation enhancers also include, e.g., one or more monounsaturated fatty acid, or a derivative thereof, including, without limitation, one or more of 5-dodecenoic acid (C12:1), 7-tetradecenoic acid (14:1), palmitoleic acid (16:1), oleic acid (C18:1), vaccenic acid (C18:1), elaidic acid (C18:1), paullinic acid (C20:1), gondoic acid (C20:1), erucic acid (C22:1), 15-docosenoic acid (C22:1), 17-tetracosenoic acid (24:1), nervonic acid (C24:1), and ximenic acid (C26:1), or one or more derivative thereof. Fatty acid or alcohol permeation enhancers further include, e.g., one or more of oleic acid (“OA”, C18:1) and oleic acid derivatives. Oleic acid derivatives may include, e.g., one or more of ethyl oleate (OA ethyl ester), oleyl oleate (OA oleyl ester), glyceryl oleate (OA glyceryl ester), sorbitan monooleate (sorbitan oleate, Span 80), and oleyl alcohol (cis-9-octadecen-1-ol). Fatty acid or alcohol permeation enhancers still further include, e.g., one or more of polyunsaturated fatty acid, and a polyunsaturated fatty acid derivative; and the polyunsaturated acids may include, without limitation, one or more of: hexadecatrienoic acid (16:3), linoleic acid (C18:2), rumenic acid (C18:2), alpha-linolenic acid (C18:3), gamma-linolenic acid (C18:3), calendic acid (C18:3), stearidonic acid (C18:4) mead acid (C20:3), eicosadienoic acid (C20:3), eicosatrienoic acid (C20:3), dihomo-gamma-linolenic acid (C20:3), arachidonic acid (C20:4), docosadienoic acid (C22:2), adrenic acid (C22:4), osbond acid (C22:5), tetracosatetraenoic acid (C24:4), tetracosapentaenoic acid (C24:5), and derivatives thereof, including without limitation, one or more of alcohols and esters, e.g. linoleyl alcohol (the fatty alcohol of linoleic acid). Fatty acid or alcohol permeation enhancers yet further include, e.g., one or more of saturated fatty acids, and saturated fatty acids derivatives; the saturated fatty acids may include, without limitation, one or more of: stearic acid (C18:0), palmitic acid (C16:0), myristic acid (C14:0), and lauric acid (C12:0). Fatty acid or alcohol permeation enhancers also include, e.g., one or more fatty acid ester, fatty acid ester derivative, and fatty acid derivative; these may include, without limitation, one or more of: ethyl oleate, methyl oleate, decyloleate, glyceryl monooleate, oleyl oleate, isopropyl palmitate (ester of isopropyl alcohol and palmitic acid), myristate, isopropyl myristate, methyl laurate (lauric acid methyl ester), glyceryl laurate (lauric acid glyceryl ester, monolaurin, glycerol monolaurate), propylene glycol monolaurate type I, propylene glycol monolaurate type II (e.g. Lauroglycol™90, commercially available from Gattefosse, Lyon, France), lauryl lactate (ester of lauryl alcohol and lactic acid), and butyl acetate. Alternatively or additionally, the fatty acid or alcohol permeation enhancers may be provided in form of an oil, or an enriched part/fraction of an oil, e.g. a plant-derived oil, that is rich in one or more fatty acid or alcohol. For example, the oil may contain, without limitation, one or more fatty acid, monounsaturated fatty acid, and polyunsaturated fatty acid. An enriched fraction of such an oil that contains fatty acid or alcohol of interest may be formed and used. Oils with suitable fatty acids include, without limitation, olive oil, macadamia oil, rapeseed oil, wall flower seed oil, mustard seed oil, nutmeg, palm oil, and coconut oil. Suitable oil fractions may include an “MCT oil” or “LCT” oil enriched e.g. in one or more of C8, C10, C12, C14, C16, C18 fatty acids.

Fatty alcohol permeation enhancers may include, without limitation, one or more saturated, monounsaturated or polyunsaturated fatty alcohol; which may include, without limitation, one or more of: butanol (C4), butyl alcohol (C4), tert-butyl alcohol (C4), tert-amyl alcohol (C5), 3-Methyl-3-pentanol (C6), capryl alcohol (C8), pelargonic alcohol (C9), capric alcohol (C10), Undecyl alcohol (C11), Lauryl alcohol (C12), Tridecyl alcohol (C13), Myristyl alcohol (C14), Pentadecyl alcohol (C15), Cetyl alcohol (C16), Palmitoleyl alcohol (cis-9-hexadecen-1-ol, C16H32O), Heptadecyl alcohol (1-n-heptadecanol, C17H36O), Stearyl alcohol (C18:0), Oleyl alcohol (C18H36O, C18:1), linoleyl alcohol (C18H340, cis,cis-9,12-Octadecadien-1-ol), Nonadecyl alcohol (C19), Arachidyl alcohol (C20H42O), octyldodecanol (C20H42O, 2-Octyldodecan-1-ol), Heneicosyl alcohol (C21), Behenyl alcohol (C22H460), Erucyl alcohol (cis-13-docosen-1-ol, C22H440), Lignoceryl alcohol (C24), and Ceryl alcohol (C26). Saturated fatty alcohol permeation enhancers may include, without limitation, one or more of: lauryl alcohol (C12), isolauryl alcohol (C12, 10-methyl-1-hendecanol), anteisolauryl alcohol (C12, 9-methyl-1-hendecanol), myristyl alcohol (C14), isomyristyl alcohol (C14, 12-methyl-1-tridecanol), anteisomyristyl alcohol (C14, 11-methyl-1-tridecanol), cetyl alcohol (C16), isopalmityl alcohol (C16, 14-methyl-1-pentadecanol), anteisopalmityl alcohol (C16, 13-methyl-1-pentadecanol), stearyl alcohol (C18), isostearyl alcohol (C18, 16-methyl-1-heptadecanol), and anteisostearyl alcohol (C18, 15-methyl-1-pentadecanol).

Fatty alcohol or acid permeation enhancers with a longer carbon chain length may be preferred for their non-irritant or skin protective effect when present in formulations for use in the methods described herein; these include e.g., without limitation, C12-C26 fatty alcohols or acids as hereinabove described, preferably C12-C18 fatty alcohols or acids as hereinabove described, and may include saturated, monounsaturated or polyunsaturated alcohols or acids. These may be combined with shorter chain permeation enhancers wherein the fatty acid/alcohol or fatty acid/alcohol moiety has a carbon chain length of C4 to C10 (i.e. C4, C5, C6, C7, C8, C10, or combinations thereof); for example, one or more of butyric acid (C4:0), isobutyric acid (C4:0), valeric acid (C5:0), isovaleric acid (C5:0), levulinic acid (C5:0), caproic acid (C6:0), caprylic acid (C8:0), capric acid (C10:0), butanol, butyl alcohol, 2-butanol, isobutanol, tert-butanol. These shorter length fatty alcohol or acid permeation enhancers may preferably be included in a smaller amount than the longer ones, e.g. from about 1% to about 10% for the longer chain enhancers, and from about 0.1% to about 5% for the shorter chain enhancers, more preferably from about 0.5% to about 2%, e.g. from about 0.5% to about 1%.

Preferred fatty alcohol or acid permeation enhancers for the formulations described herein may include, without limitation, one or more of: oleic acid, ethyl oleate (OA ethyl ester), oleyl oleate (OA oleyl ester, C36H6802), glyceryl oleate (OA glyceryl ester), sorbitan monooleate (sorbitan oleate, Span 80), and oleyl alcohol (cis-9-octadecen-1-ol), elaidic acid (C18:1), gondoic acid (C20:1), erucic acid (C22:1), nervonic acid (C24:1), and ximenic acid (C26:1), or one or more derivative thereof. Polyunsaturated acids such as hexadecatrienoic acid (16:3), linoleic acid (C18:2), alpha-linolenic acid (C18:3), gamma-linolenic acid (C18:3), calendic acid (C18:3), stearidonic acid (C18:4) mead acid (C20:3), eicosadienoic acid (C20:3), eicosatrienoic acid (C20:3), dihomo-gamma-linolenic acid (C20:3), arachidonic acid (C20:4), docosadienoic acid (C22:2), and derivatives thereof, including without limitation, alcohols and esters, e.g. linoleyl alcohol (the fatty alcohol of linoleic acid).

It is preferred that the permeation or penetration enhancer (or combination thereof) be non-irritating to human skin for the duration of use, or be used in an amount that is non-irritating for the duration of use, in particular when the use is in form of an in-adhesive patch over multiple days. Many known penetration enhancers are irritating to human skin, especially when used for a prolonged period and especially when used in form of an occlusive or semi-occlusive patch (rather than e.g. a topical application such as e.g. a lotion). As will be apparent to the skilled person that the amount of the optional penetration enhancer should be sufficiently low to avoid such irritation. In formulations according to the invention, it is preferred to exclude any skin-irritating penetration enhancers or other skin-irritating excipients, and if used, it is preferred to include them only in a low non-irritating amount.

The patch formulation may comprise one or more plasticizer to avoid brittleness and impart flexibility to the adhesive matrix layer. The necessity and choice of plasticizer will depend on the particular adhesive and formulation. Suitable plasticizers are well known in the art. For example, without limitation, the one or more optional plasticizer may be selected from, without limitation, one or more of: glycols (in particular, without limitation, e.g. polyethylene glycol 400, polyethylene glycol 600, propylene glycol), higher alcohols (e.g. dodecanol), surfactants, sebacic acid esters (e.g. dibutyl sebacate, diethyl sebacate), citric acid esters (e.g. tributyl citrate, triethyl citrate), phthalic acid esters (e.g. diethyl phthalate, dibutyl phthalate), glycerol or glycerol esters (e.g. glycerine triacetate, glycerin), sugar alcohols (e.g. sorbitol, sucrose), tartaric acid esters (e.g. diethyl tartrate), oil (e.g. silicone oil, mineral oil), triacetin, and diisopropyl adipate. For inclusion into an adhesive patch formulation, and in particular an acrylic PSA patch formulation, preferred plasticizers include, without limitation, one or more of glycerol and glycerol esters. Further plasticizers may be found in “Handbook of Plasticizers” by George Wypych, 2004, Chem Tec Publishing), which is hereby incorporated by reference in its entirety.

Further optional excipients include for example, without limitation, one or more pH adjusting agents selected from, without limitation, buffers (e.g. citrate buffer, phosphate buffer, acetate buffer), acids (e.g. hydrochloric acid, acetic acid, succinic acid, citric acid, ascorbic acid, phosphoric acid), bases (e.g. sodium bicarbonate, triethanolamine, sodium hydroxide, calcium hydroxide, potassium hydroxide, ammonium hydroxide), and tromethamine. Preferred pH adjusting agents include, without limitation, one or more of sodium bicarbonate, sodium carbonate, acetic acid, and ascorbic acid. The pH adjusting/buffering agent or stabilizer helps to maintain the appropriate pH of the transdermal formulation, preferably in the range of pH 4.0-8.0, more preferably 5-7, most preferably 6-6.8. The amount will be chosen depending on the type and strength of the agent as will be apparent to a person of ordinary skill, e.g. from about 0.01% to about 30% w/w.

Still further optional excipients include for example, without limitation, one or more of emulsifying agents, auxillary emulsifying agents, surfactants, suspending agents, preservatives, antioxidants, chelating agents, emollients, humectants, demulcents, skin irritation reducing agents, tackifiers, fillers, cross-linking agents, resins, crystallization inhibitors, and clays. For illustrative purposes, examples for various optional excipients that may be suitable additives in a patch formulation, in particular an acrylic PSA patch formulation, are given below.

Such optional emulsifying agents, auxiliary emulsifying agents, surfactants and suspending agents may include, without limitation, one or more of monoglycerides, diglycerides, polyoxyl stearate, a mixture of triceteareth-4 phosphate with ethylene glycol palmitostearate and with diethylene glycol palmitostearate, polyglyceryl-3 diisostearate, a mixture of PEG-6 stearate with ethylene glycol palmitostearate and with PEG-32 stearate, oleoylpolyoxyl-6 glycerides, lauroyl polyoxyl-6 glycerides, caprylocaproyl polyoxyl-8 glycerides, propylene glycol monocaprylate type I, propylene glycol monolaurate type II, propylene glycol monolaurate type I, propylene glycol monocaprylate type II, polyglyceryl-3 dioleate, a mixture of PEG-6 stearate with PEG-32 stearate, lecithin, cetyl alcohol, cholesterol, bentonite, veegum, magnesium hydroxide, dioctyl sodium sulfosuccinate, sodium lauryl sulfate, triethanolamine stearate, potassium laurate, polyoxyethylene fatty alcohol ethers, glyceryl monostearate, polyoxyethylenepoloxypropylene block copolymers (poloxamers), sorbitan monolaurate, lanolin alcohols and ethoxylated lanolin alcohols, sorbitan fatty acid esters, sucrose distearate, sodium alginate, alginic acid, hectorite, and aluminum silicate.

Emollients, humefactant, demulcents and skin irritation reducing agents may be selected from, without limitation, one or more of glycerin, propylene glycol, mineral oil, petrolatum, lanolin, paraffin, cetyl alcohol, cetyl esters wax, zinc oxide, and dimethicone.

Preservatives and antioxidants may be selected from, without limitation, one or more of sodium metabisulfite, citric acid, ascorbic acid, BHA, BHT, butylated hydroxyanisole, butylated toluene, alpha tocopherol, acorbyl palmitate, propionic acid, sodium bisulfate, propyl gallate, monothioglycerol, ascorbic acid, sodium ascorbate, benzethoniumchloride, chlorhexidine, phenylethyl alcohol, chloroxylenol, cresol, hexetidine, phenoxyethanol, chlorobutanol, ascorbic acid, benzoic acid, sorbic acid, potassium sorbate, potassium metabisulfite, sodium metabisulfate, phenol, potassium benzoate, dehydroacetic acid, cetylpyridinium chloride, parabens, benzyl alcohol, benzalkonium chloride).

Chelating agents may be selected from, without limitation, one or more of sodium edetate, edetic acid, tartaric acid, fumaric acid, disodium edetate, trisodium edetate, dipotassium edetate).

Fillers may be selected from, without limitation, one or more of lactose, magnesium stearate, mannitol, titanium dioxide, talc, shellac, colloidal silicone dioxide, kaolin etc.).

Cross-linking agents may be selected from, without limitation, one or more of melamine formaldehyde (Aerotex® M3, Aerotex® 3730).

Resins may be selected from, without limitation, one or more of polyamide resin (Versamid® 100 P77.5, Versamid® 100 X-65).

Many suitable methods and corresponding materials to make the patches described herein are known in the art. According to an embodiment of the present invention, a patch may be formed, for example, without limitation, by solvent casting onto a backing layer or release liner, and sandwiching between both, as described herein.

Many suitable materials for the backing layer and release liner are known, and include polymer films, fabrics and non-woven materials, e.g. continuous films that prevent ingress of external moisture into the adhesive layer from activities such as showering or bathing. The backing and release liner should preferably be occlusive, or substantially occlusive. Such films include, without limitation, polypropylene, polyvinyl chloride, cellulose acetate, ethyl cellulose, polyurethane, polyethylene, and polyester. Optionally, the backing may be a layered composite that include a metal, such as, without limitation aluminum, e.g. polyethylene terephthalate-aluminium-polyethylene composites, or e.g. a polyester and an ethylene vinyl acetate copolymer heat seal layer (particularly as a backing), or e.g. a fluoropolymer coated polyester film (particularly as a release liner. Suitable backing layers include, without limitation, Scotchpak 1006, 1022, 1109, 9723, 9732, 9733 (3M company); suitable release liners include, without limitation, Scotchpak 1006, 9709, 9741, 9742, 9744, and 9755 (3M company). The thickness of the backing layer and of the release liner is generally more than 10 μm and less than 200 μm, typically about 20 μm to about 120 μm, e.g. about 40 μm to about 100 μm.

As used herein, the term “pharmaceutically acceptable salts” includes acid addition salts or addition salts of free bases. The term “pharmaceutically acceptable salts” within its scope include each of all the possible isomers and their mixtures, and any pharmaceutically acceptable metabolite, bioprecursor and/or pro-drug, such as, for example, a compound which has a structural formula different from the one of the compounds recited or described, yet is directly or indirectly converted in vivo into such a compound upon administration to a subject, such as a mammal, and particularly a human being.

As used herein, the terms “subject” and “patient” are used interchangeably and include mammals; these may be non-primate mammals (e.g., without limitation, cows, pigs, horses, cats, dogs, and rats) and primate mammals (e.g., without limitation, monkeys and humans). Non-human animals include farm animals (e.g. horses, pigs, and cows) and pets (e.g. dogs and cats). In specific embodiments, the subject may be a human patient, or a non-human patient.

As used herein, the term “active”, “agent”, or “therapeutic agent” refers to any molecule, compound, methodology and/or substance that is used for the prevention, treatment, management and/or diagnosis of a disease, disorder or condition.

As used herein, the term “effective amount” refers to the amount of a therapy or agent that is sufficient to result in the prevention of the development, recurrence, or onset of a disease or condition, the prevention, treatment, reduction or amelioration of one or more symptoms thereof, the enhancement or improvement of the prophylactic effect(s) of another therapy, the reduction of the severity or the duration of a disease or condition, the amelioration of one or more symptoms of a disease or condition, the prevention of the advancement of a disease or condition, the regression of a disease or condition or one or more of its symptoms, and/or the enhancement or improvement of the therapeutic effect(s) of another therapy.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “compounds of the present invention” or “compounds for use in the present invention” (unless specifically identified otherwise) refers to a PARP-inhibitor active and an olanzapine active, administered in a common administration scheme.

As used herein, the term ““treat”, “treating”, “treatment”, or “therapy” of a disease or disorder refers to ameliorating the disease or disorder; for example slowing, arresting or reducing the disease, its development, or one or more clinical symptom thereof; the term also refers to alleviating or ameliorating one or more physical parameter, whether or not discernible by the patient; the term also refers to physically and/or physiologically modulating the disease or disorder (e.g. by stabilization of a discernible symptom and/or physical parameter).

As used herein, the term “prevention” of a disease or disorder refers to the administration of the compounds of the invention to a subject before any symptoms of that disease or disorder are apparent.

As used herein, the term “disease” or “disorder” includes symptoms (or physical or physiological parameters) associated with adverse effects or side effects that occur upon administration of an active, in particular: a PARP-inhibitor, to a patient. Such symptoms specifically include nausea and/or vomiting.

As used herein, a patient or subject is “in need of” a treatment if the patient or subject would benefit biologically, medically or in quality of life from such treatment.

The term “derivative” or “derivatized” as used herein includes chemical modification of a compound, or pharmaceutically acceptable salts thereof or mixtures thereof. That is, a “derivative” may be a functional equivalent of a compound which is capable of inducing the functional activity of the compound in a given subject or application.

As used herein, the terms “composition” and “formulation” may be used interchangeably, unless otherwise indicated. Generally, a formulation may be used as a stand-alone non-occlusive transdermal composition for application to the skin, or may be used in form of or to prepare a transdermal patch for application to the skin (patch formulation).

As used herein, the term “topical delivery” means delivery of drug into systemic circulation through the skin, which includes occlusive and non-occlusive delivery by a transdermal composition (typically non-occlusive) or patch (typically occlusive, depending on the backing layer of the patch).

As used herein, the term “a,” “an,” “the” and similar terms used in the context of the present invention are to be construed to cover both the singular and plural unless otherwise indicated herein or clearly contradicted by the context. The use of any and all examples, or exemplary language (e.g. “such as”, “for example”, “illustrative”, “e.g.”) provided herein is intended merely to better illustrate the invention and is not intended to limit the scope of the invention.

### EXAMPLES

### Example 1: Common Oral Administration Scheme

A PARP-inhibitor (one or more of olaparib, rucaparib, and niraparib) is administered to a patient two or three times daily in form of a tablet, at a daily dose of about 600 mg (e.g. two tablets of 150 mg twice, one tablet of 300 mg once, or 200 mg three times). The dose may be reduced to adjust to the individual as necessary. With each dose of a PARP-inhibitor, a tablet of olanzapine is administered (5 mg or 10 mg), to provide a total daily dose of 10-15 mg olanzapine. Thus, nausea and vomiting is reduced.

### Example 2: Common Oral/Transdermal Administration Scheme

A PARP-inhibitor (one or more of olaparib, rucaparib, and niraparib) is administered as described in example 1. At the time of first administration of the PARP-inhibitor, or 1-6 hours beforehand, an olanzapine patch is applied to the skin of the patient. The patch is chosen depending on the duration of PARP-inhibitor administration. For example, a patch may provide delivery for 1-7 days, and is chosen accordingly. The patch can be removed in case PARP-administration is terminated early.

### Example 3: Forming an Olanzapine Coating Formulation

An olanzapine formulation is prepared by mixing olanzapine base, one or more enhancer (e.g. one or more of oleic acid and isopropyl myristate), optionally lactic acid, and Diethylene glycol monoethyl ether (DEGEE, e.g. Transcutol®).

| Coating formulations | 1 | 2 | 3 | 4 | 5 | 6 |
|---|---|---|---|---|---|---|
| OLA base | 15 | 15 | 15 | 15 | 15 | 15 |
| DMSO | 31 | 31 | 31 | 31 | 31 | 31 |
| LA | 31 | 31 | — | — | 31 | — |
| Oleic Acid | 10 | — | 10 | — | 5 | 5 |
| IPM | — | 10 | — | 10 | 5 | 5 |
| DEED | 13 | 13 | 44 | 44 | 13 | 44 |
| Total | 100 | 100 | 100 | 100 | 100 | 100 |

Each formulation may be tested by in-vitro skin permeability test using modified franz-diffusion cells with human cadaver skin as a diffusion barrier. Phosphate Buffered Saline (PBS) of pH 7.4 with 0.01% sodium azide is used as a receiving medium to determine skin flux. The experiments are performed at 32 DEG.C (body temperature). Samples are collected at predetermined time intervals and the drug is quantified by reversed phase HPLC analysis.

### Example 4: Forming Olanzapine Patches

A patch may be formed from the by solvent casting an olanzapine formulation onto a backing layer or release liner, and sandwiching between both. Patches may be formed by adding a polymer, preferably a polyacrylic polymer, and mixing until a uniform coating formulation is obtained. The coating formulation may be solvent cast onto a backing layer or release liner, and sandwiched between both. A coating formulation may be prepared as detailed in example 3; optionally, a surfactant and/or sodium bicarbonate (to adjust the pH) are added. Optionally, to the resulting coating solution, the polymer Hydroxypropyl cellulose acetate (AquaSolve™ HPMCAS MF, Ashland, Covington, Ky., U.S.A.) is added, e.g. in an amount of 5% by dry weight of the total, and the mixture is homogenized to form a viscous gel. The coating formulation or gel is mixed with the a polyacrylic adhesive (e.g. Duro-Tak® 387-2516), in a ratio of about 1:1 by weight (i.e. 50% coating formulation/gel and 50% adhesive, see table below) and stirred until it forms a uniform mixture. The resulting mixture is cast with a 0.2 mm coating thickness onto a suitable support (typically directly onto a backing layer or release liner) using a scalpel. A suitable backing layer is Scotchpak 9733 (3M company), alternatively, for use as release liner, Scotchpak 9744 (3M company) can be employed. The patches are dried at 70° C. for 30 min to achieve 50 mg/cm2 dried weight.

| | | Coating formulations | Amphiphilic |
|---|---|---|---|
| Patch | Adhesive | 1-6 | Polymer |
| 1-6a | 50 | 50 | — |
| 1-6b | 50 | 45 | 5 |
| Total | 100 | 100 | 100 |

The formed patches may be tested using franz-diffusion cells as described above.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature rather than restrictive.

1. A method of treating nausea or vomiting in a human subject, the method comprising the steps of:
administering a therapeutic amount of a PARP-inhibitor to the human subject in need thereof; and
administering olanzapine to the human subject in an amount sufficient to treat one or more of nausea and vomiting;
wherein administering the PARP-inhibitor and olanzapine are performed as part of a common administration scheme,
wherein the common administration scheme is characterized by administering olanzapine about 1 to about 24 hours before administration of the PARP-inhibitor.

2. (canceled)

3. The method of claim 1, wherein the common administration scheme is characterized by co-administering olanzapine and the PARP-inhibitor within a window of time of 1 hour or less.

4. A method of treating nausea or vomiting in a human subject, the method comprising the steps of:
administering a PARP-inhibitor to the human subject;
administering olanzapine to the human subject;
within a first predetermined amount of time, achieving a minimum level of efficacy for treating nausea or vomiting in the human subject;
within a second predetermined amount of time, achieving a preferred level of efficacy for treating nausea or vomiting in the human subject; and
maintaining the preferred level of efficacy within a predetermined range for treating nausea or vomiting in the human subject for a third predetermined amount of time.

5. The method of claim 4, wherein the first predetermined amount of time is less than about an hour.

6. The method of claim 4, wherein the first predetermined amount of time is less than about thirty minutes.

7. The method of claim 4, wherein the second predetermined amount of time is less than about 5 hours.

8. The method of claim 4, wherein the second predetermined amount of time is less than about 3 hours.

9. The method of claim 4, wherein the third predetermined amount of time is at least 2 days.

10. The method of claim 4, wherein the third predetermined amount of time is at least 5 days.

11. The method of claim 4, wherein the third predetermined amount of time is at least 7 days.

12. The method of claim 4, wherein the third predetermined amount of time is at least 14 days.

13. The method of claim 4, wherein the minimum level of efficacy is achieved when the human subject blood serum level of olanzapine is at least 10 ng/ml.

14. The method of claim 4, wherein the preferred level of efficacy is achieved when the human subject blood serum level of olanzapine is at least 20 mg/l.

15. The method of claim 4, wherein the predetermined range is a blood serum level of olanzapine that is between about 20 ng/ml. and about 40 ng/ml.

16. The method of claim 4, wherein the nausea or vomiting in a human subject is induced by the administration of the PARP-inhibitor.
